(19) United States
(12) Patent Application Publication
(10) Pub. No.: US 2017/0176418 A1
(43) Pub. Date: Jun. 22, 2017

(54) DEVICE FOR DETECTION OF DISEASE STATES AND APPLICATIONS OF SAME

(71) Applicant: VIGILANT BIOSCIENCES, INC., Ft. Lauderdale, FL (US)

(72) Inventors: Robert C. Bohannon, Elkhart, IN (US); Seven C. Bohannon, Granger, IN (US); Matthew H.J. Kim, Parkland, FL (US)

(21) Appl. No.: 15/127,156

(22) PCT Filed: Mar. 19, 2015
(86) PCT No.: PCT/US2015/021532

§ 371 Date: Sep. 19, 2016

Related U.S. Application Data
(60) Provisional application No. 61/955,629, filed on Mar. 19, 2014.

(51) Int. Cl. G01N 33/52; G01N 21/84; G01N 33/543; G01N 33/558; G01N 33/68

(52) U.S. Cl. CPC G01N 33/523; G01N 33/558; G01N 33/6827; G01N 33/54386; G01N 21/8483; G01N 2333/70585

(57) ABSTRACT

A device for testing a biological sample includes a test strip. The test strip includes a substrate having a first surface and an opposite, second surface; a first sample pad and a second sample pad disposed on the first surface and the second surface respectively, and configured to receive the biological sample; a first test pad disposed on the first surface, in contact with the first sample pad, and configured to test the biological sample received from the first sample pad; and a second test pad disposed on the second surface, in contact with the second sample pad, and configured to test the biological sample received from the second sample pad. The first and second sample pads may be configured to evaluate the CD44 protein and total protein of the sample, and used for early detection of neck squamous cell carcinoma (HNSCC).

### CROSS-REFERENCE TO RELATED PATENT APPLICATIONS

This PCT application claims priority and the benefit of U.S. Provisional Application Ser. No. 61/955,629, filed on Mar. 19, 2014, entitled “DEVICE FOR EARLY DETECTION OF DISEASE STATES,” by Robert C. Bohannon, Seven C. Bohannon, and Matthew H. J. Kim, the disclosure of which is incorporated herein in its entirety by reference.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The disclosure relates generally to a device for detecting states of diseases, and more specifically to a device having one or more strips for detection of disease states and applications of the same.

### BACKGROUND OF THE DISCLOSURE

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the disclosure.

Detection of disease states of an object may involve performing multiple independent or related tests to the object. Each test may determine or evaluate at least one character of the object.

Cancers are among the leading causes of death worldwide. Early detection of cancers, such as head and neck squamous cell carcinoma (HNSCC), is essential for efficient prevention and treatment of the cancers. However, fast, cost-effective, and accurate early detection of cancers is still a challenge.

Therefore, a heretofore unaddressed need exists in the art to address the aforementioned deficiencies and inadequacies.

### SUMMARY OF THE DISCLOSURE

In one aspect, the disclosure is directed to a device for testing a biological sample.

In one embodiment, the device includes a test strip. The test strip includes a substrate having a first surface and an opposite, second surface, a first sample pad and a second sample pad disposed on the first surface and the second surface respectively and configured to receive the biological sample, a first test pad disposed on the first surface of the substrate, in contact with the first sample pad, and configured to test the biological sample received from the first sample pad, and a second test pad disposed on the second surface of the substrate, in contact with the second sample pad, and configured to test the biological sample received from the second sample pad.

In one embodiment, the first test pad is configured to determined presence or a concentration of a target molecule of interest in the biological sample, and the second test pad is configured to determine presence or a concentration of total protein in the biological sample. In one embodiment, the target molecule of interest is a biomarker for indicating a biological state or a condition. In one embodiment, the biomarker includes CD44 or a protein homologous to CD44.

In certain embodiments, the presence of the target molecule of interest and the presence of the total protein in the biological sample are determined by visually viewing the first test pad and the second test pad respectively.

In one embodiment, the concentration of the target molecule of interest and the concentration of the total protein in the biological sample are determined using an electronic reader or a reflectance reader.

In one embodiment, the first test pad includes a test line and a control line, the device further includes a target intensity chart including a relationship between a color intensity of the test line and the concentration of the target molecule of interest, and the concentration of the target molecule of interest in the biological sample is determined by comparing the color intensity of the test line with the colored chart.

In one embodiment, the device further includes a total protein intensity chart including a relationship between a color intensity of the second test pad and the concentration of the total protein, and the concentration of the total protein in the biological sample is determined by comparing the color intensity of the second test pad with the total protein intensity chart. In one embodiment, the second test pad is configured to change color when the concentration of the total protein is higher than a predetermined concentration.

In one embodiment, the test strip further includes a first sink pad disposed on the first surface of the substrate such that the first test pad is placed between the first sample pad and the first sink pad.

In one embodiment, the device further includes a container configured to accommodate the biological sample, wherein the test strip is placed in the container for receiving the biological sample.

In one embodiment, the device disposable or is single use device.

In one embodiment, the substrate is flexible and formed of plastic, metal, or glass, and at least one of the first test pad and the second test pad is formed of a nitrocellulose membrane, a nylon membrane, or an activated cellulose membrane.

In one embodiment, the substrate includes a first substrate sheet and a second substrate sheet attached back to back, such that the first substrate sheet exposes the first surface and the second substrate exposes the second surface.

In another aspect, the disclosure relates to a device for testing at least one biological sample. In one embodiment, the device includes a support member having multiple side surfaces, and multiple test strips. Each test strip is attached to one of the side surfaces of the support member. At least one of the test strips includes a sample pad configured to receive the biological sample, and a test pad in contact with the sample pad, and configured to test the at least one biological sample received from the sample pad.

In one embodiment, the support member is in a shape of prism comprising triangular prism, square prism, cube, pentagonal prism, and a prism having more than five side surfaces.

In one embodiment, the support member has a hollow structure, such that each of the side surfaces comprises an outer surface facing outward and an opposite, inner surface facing inward.

In one embodiment, at least one of the side surfaces has a first test strip of the multiple test strips disposed on the outer surface of the at least one of the side surfaces, and a second test strip of the multiple test strips disposed on the inner surface of the at least one of the side surfaces.

In one embodiment, the first test strip is configured to determine presence or a concentration of CD44 protein in the at least one biological sample, and the second test strip is configured to determine presence or a concentration of total protein in the at least one biological sample.

In one embodiment, a first test strip of the multiple test strips is disposed on at least one surface of the side surfaces, and configured to determine presence or a concentration of CD44 protein in the at least one biological sample, and a second test strip of the multiple test strips is disposed on at least the other one of the side surfaces, and configured to determine presence or a concentration of total protein in the at least one biological sample.

In one embodiment, the support member includes a plurality of notched receptacles. Each notched receptacle has a sealed end and an open end. Each of the test strips is slidably received in one of the notched receptacles through the open end thereof.

In one embodiment, at least one of the test strips further includes a substrate attached to one of the side surfaces, and at least one of the test strips further includes at least one sink pad in contact with the test pad of the at least one of the test strips.

In one embodiment, the substrate is flexible and formed of plastic, metal, glass, or the like. In one embodiment, the test pad is formed of a nitrocellulose membrane, a nylon membrane, or an activated cellulose membrane.

In one embodiment, the device further includes a container configured to accommodate the at least one biological sample, wherein the support member and the test strips are placed in the container for receiving the biological sample.

In one embodiment, the device is disposable or is a single use device.

In one aspect, the disclosure relates to a kit for detection of diseases including cancers. The kit comprises at least one device disclosed above.

Further areas of applicability of the disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The following figures form part of the present specification and are included to further demonstrate certain aspects of the disclosure. The disclosure may be better understood by reference to one or more of these figures in combination with the detailed description of specific embodiments presented herein. The drawings described below are for illustration purposes only. The drawings are not intended to limit the scope of the present teachings in any way.

FIG. 1 shows a side sectional view of a device according to one embodiment of the disclosure.

FIG. 2 shows a side sectional view of a device according to one embodiment of the disclosure, where the device has two test strips.

FIG. 3 shows a side sectional view of a device according to one embodiment of the invention, where the device has two test strips.

FIG. 4A shows schematically a perspective view of a device according to one embodiment of the disclosure.

FIG. 4B shows schematically a front view of a support member and the test strips received in the support member according to certain embodiments of the disclosure.

FIGS. 5A-5D show schematically cross-section views of devices according to certain embodiments of the disclosure, where the test strips are not assembled to the support member.

FIGS. 6A-6D show schematically cross-section views of devices according to certain embodiments of the disclosure, where the test strips are assembled to the support member.

### DETAILED DESCRIPTION OF THE DISCLOSURE

The disclosure will now be described more fully hereinafter with reference to the accompanying drawings, in which exemplary embodiments of the disclosure are shown. This disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the disclosure or of any exemplified term. Likewise, the disclosure is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the disclosure.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the disclosure, and will not be interpreted in an idealized or overly formal sense unless expressly so defined herein.

As used herein, “around”, “about”, “substantially” or “approximately” shall generally mean within 20 percent, preferably within 10 percent, and more preferably within 5 percent of a given value or range. Numerical quantities given herein are approximate, meaning that the terms “around”, “about”, “substantially” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

The description will be made as to the embodiments of the disclosure in conjunction with the accompanying drawings. In accordance with the purposes of this disclosure, as embodied and broadly described herein, this disclosure, in one aspect, relates to a device for testing a biological sample.

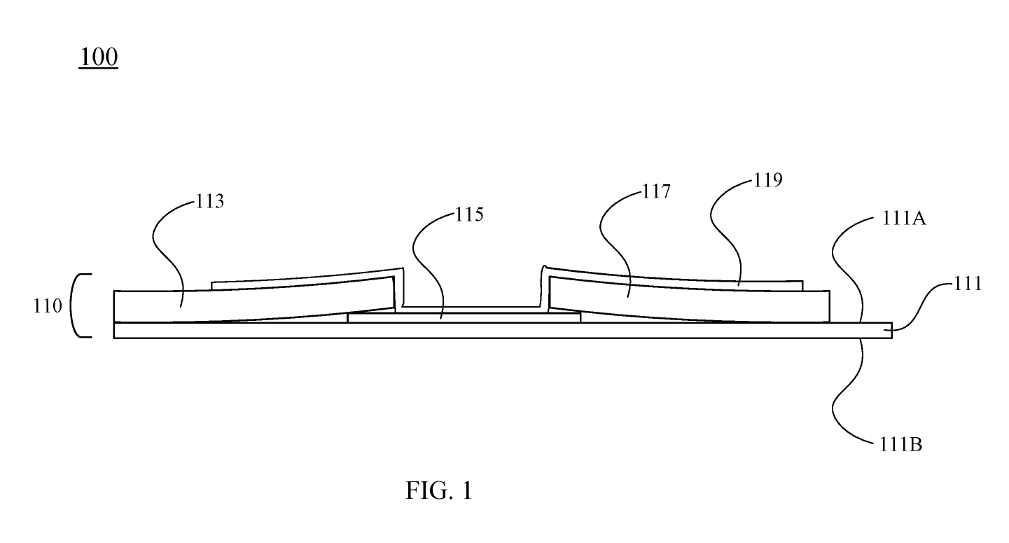

In one embodiment, the device includes a test strip. FIG. 1 shows a side sectional view of a test strip according to one embodiment of the disclosure. As shown in FIG. 1, the test strip 110 includes a substrate 111, a sample pad 113, a test pad 115, a sink pad 117, and a mask layer 119.

The substrate 111 is used for providing support for the sample pad 113 and the test pad 115. In certain embodiments, the substrate 111 may be formed from a flexible material with sufficient strength, such that the strip 110 is not easily broken during shipment, storage, and operation. The material of the substrate 111 may be plastic, paper, metal, glass, or the like. In one embodiment, the substrate 111 is made of a G & L plastic-backed card (Lohmann Corporation, Orange, Va., US). The substrate 111 has a first surface 111A and an opposite, second surface 111B. In one embodiment, the substrate 111 has single-sided adhesive. In one embodiment, the substrate 111 has double-sided adhesive. The adhesive first surface 111A provides attachment force for the sample pad 113, the test pad 115, and the sink pad 117 to be disposed thereon. The second surface 111B, optionally adhesive, may provide attachment force for attaching at least one of another substrate, another sample pad, another test pad, or another sink pad.

In certain embodiments, the substrate 111 has a length of about 20-300 millimeter (mm), and a width of about 1-20 mm. In one embodiment, the substrate 111 has a length of about 40-160 mm, and a width of about 3-10 mm. In one embodiment, the substrate 111 has a length of about 80 mm, and a width of about 4.7 mm.

The sample pad 113 is disposed on the first surface 111A of the substrate 111 and configured to receive the biological sample, and transfer at least a portion of the received biological sample to the test pad 115. In one embodiment, the sample pad 113 is typically treated with surfactants, proteins, etc. In one embodiment, the sample pad 113 includes colored latex or anything that can be visually or electronically detected that serves as labeled material. The labeled material may be applied with a conjugate diluent or a stabilizing agent, such as sucrose, surfactants, buffers, proteins, and sometimes salts. The treatment by the conjugate diluents or the stabilizing agents is important to make the test work properly with the sample being tested. In certain embodiments, the sample pad 113 includes a sampling portion and a conjugating portion. The sampling portion is the left portion (or the end portion, or the outside portion) for receiving the sample, and the conjugating portion is the right portion (or the inner side portion) which is in contact with the test pad 115. The conjugating portion includes conjugates. For example, the conjugating portion of the sample pad 113 may include a detector antibody as the conjugates for specifically binding a target antigen. In one embodiment, the sample pad 113 contains at least one of BMS209-detector antibody, colloidal 526 gold, and colloidal Gold Blocker, colored latex, or a material that can be electronically detected. In certain embodiments, the sample pad 113 may include two separated pads, i.e., a sampling pad for receiving a sample and a conjugating pad having conjugates. In certain embodiments, the sample pad 113 is formed of a glass fiber, such as a glass fiber Ahlstrom 8964 pad (Ahlstrom Corporation, Helsinki, Finland).

In certain embodiments, the sample pad 113 has a length of about 8-120 mm, and a width of about 1-20 mm. In one embodiment, the sample pad 113 has a length of about 15-60 mm, and a width of about 3-10 mm. In one embodiment, the sample pad 113 has a length of about 30 mm, and a width of about 4.7 mm.

The test pad 115 (or the analytical membrane, or the detection membrane) is disposed on the first surface 111A of the substrate 111 and has a portion in contact with the sample pad 113, such that the sample received by the sample pad 113 and the conjugates on the conjugating portion of the sample pad 113 may be partially migrated to the test pad 115 for being tested. The end of the conjugating portion of the sample pad 113 is disposed over one end of the test pad 115 and is in contact with the test pad 115. In certain embodiments, the sample pad 113 overlaps the test pad 115 by about 1-6 mm. In one embodiment, the overlap is about 2-3 mm. In certain embodiments, the test pad 115 is made of a nitrocellulose membrane, a nylon membrane, an activated cellulose membrane, or any other material that is capable of anchoring the capture reagent for capturing the target molecule of interest. In one embodiment, the nitrocellulose membrane is a Millipore HF120 nitrocellulose membrane (EMD Millipore, Billerica, Mass., US).

In certain embodiments, the test pad 115 includes a control line and a test line. In certain embodiments, the test line is closer to the sample pad 113 and the control line is farther to the sample pad 113, such that the sample reaches the test line first, and reaches the control line later. In certain embodiments, the test line and the control line are configured to determine presence or concentration of a target molecule of interest. The target molecule of interest may be a biomarker indicting a biological state or condition. The biomarker may be specific to a biological system such as cardiovascular system, metabolic system or the immune system, and a disease such as cancer or diabetes, etc. The biological state or condition may include the health of the biological system, the presence or states of a disease, etc. In certain embodiments, the test line includes BMS209-capture antibody, and the control line includes goat anti-mouse (GAM) antibody, and the amount of CD44 in the biological sample is indicated by the color or color intensity of the test line. In certain embodiment, the color of the test line is compared to a color intensity chart to evaluate the feature of a target molecule of interest (for example, a protein), such as the concentration of the target molecule of interest, where the color intensity chart shows a relationship between intensity of a color and the concentration of the target molecule of interest. In one embodiment, the test line may only show two different colors, i.e. as a negative result, the test line may show no color or maintain its color indicating absence of the target molecule of interest in the sample, or as a positive result, the test line may change its color indicating presence of the target of interest in the sample. In certain embodiments, the control line may show no color or maintain its original color to show a negative result, which means the failure of the test, and may change to another color to show a positive result, which means the success of the test. In certain embodiments, the intensity of the control line may also be used to extract certain information other than validating the test. In certain embodiments, the result from the test line is valid only when the control line shows a positive result. The color changes of the test line and the control line is not limited by the patterns as described above, as long as the color change pattern is consistent with the reaction mechanism, is reliable, is predictable, and has sufficient sensitivity.

In certain embodiments, a color of at least one of the test line and the control line is evaluated by an electronic reflectance measured by a reflectance reader. In one embodiment, a color of at least one of the test line and the control line is evaluated by taking a picture of the corresponding line and analyzing the picture taken. In one embodiment, the concentration of the target molecule of interest is determined using an electronic reader with a charge-coupled device (CCD) camera, a reflectance reader, or other optical methods to give quantitative results. In certain embodiments, only the intensity of the test line is analyzed, and the color change of the control line may simply be determined by a viewer or by comparing the color with a reference color. For example, the quantity of the intensity of the test line is determined for evaluating the amount of the target molecule of interest, while whether or not the color of the control line is changed or shown is used to determine the validity of the test.

In certain embodiments, the test pad 115 has a length of about 5-100 mm, and a width of about 1-20 mm. In one embodiment, the test pad 115 has a length of about 12-50 mm, and a width of about 3-10 mm. In one embodiment, the test pad 115 has a length of about 25 mm, and a width of about 4.7 mm.

The sink pad 117 (or absorbent pad) is disposed on the first surface 111A of the substrate 111, and covers an end of the test pad 115, such that one end of the test pad 115 is covered by the sample pad 113 and the other end of the test pad 115 is covered by the sink pad 117. The sink pad 117 is configured to provide a siphon effect, such that the sample received from the sample pad 113 flows to the test pad 115 and toward the sink pad 117. In one embodiment, the sink pad 117 is formed from an Ahlstrom 222 absorbent sink pad. In certain embodiments, the strip 110 may not include the sink pad 117.

In certain embodiments, the sink pad 117 has a length of about 5-100 mm, and a width of about 1-20 mm. In one embodiment, the sink pad 117 has a length of about 12-50 mm, and a width of about 3-10 mm. In one embodiment, the sink pad 117 has a length of about 25 mm, and a width of about 4.7 mm. In one embodiment, one end of the sink pad 117 in contact with the test pad 115 covers one end of the test pad 115, and the sink pad 117 overlaps the test pad 115 by about 1-6 mm. In one embodiment, the overlap is about 2-3 mm.

The mask layer 119 is covered on a portion of the top surface of the sample pad 113, the top surface of the test pad 115, and a portion of the top surface of the sink pad 117. The mask layer 119 is configured to hold the sample pad 113, the test pad 115, and the sink pad 117 in place, provides a transparent window for viewing the test line and the control line, and provides for example arrows to indicate the direction of the strip. In one embodiment, the mask layer 119 may substantially covers the conjugating portion of the sample pad 113, but not the sampling portion. In certain embodiments, the strip 110 may not include the mask layer 119.

In certain embodiments, the mask layer 119 has a length of about 15-250 mm, and a width of about 1-20 mm. In one embodiment, the mask layer 119 has a length of about 30-120 mm, and a width of about 3-10 mm. In one embodiment, the mask layer 119 has a length of about 65 mm, and a width of about 4.7 mm.

The mask layer 119 may cover a part of the sample pad 113, the whole length of the test pad 115, and a part of the sink pad 117. In one embodiment, the part of the sample pad 113 exposed out of the mask layer 119 has a length of about 2-40 mm. In one embodiment, the part of the sample pad 113 exposed out of the mask layer 119 has a length of about 5-20 mm. In one embodiment, the part of the sample pad 113 exposed out of the mask layer 119 has a length of about 10 mm. In one embodiment, the part of the sample pad 113 exposed out of the mask layer is substantially the sampling portion. In one embodiment, the part of the sink pad 117 exposed out of the mask layer 119 has a length of about 0.5-10 mm. In one embodiment, the part of the sink pad 117 exposed out of the mask layer 119 has a length of about 1-5 mm. In one embodiment, the part of the sink pad 117 exposed out of the mask layer 119 has a length of about 2 mm.

The portion of the mask layer 119 covered on the sample pad 113 and the sink pad 117 may have a color, such as a blue color. The portion of the mask layer 119 covered on the test pad 115 may be transparent, such that the control line and the test line are viewable through the transparent portion of the mask layer 119. The portion of the mask layer 119 covered on the sample pad 113 may include arrows to indicate the direction of the test strip 110. For example, the arrow may be transparent or white on the blue colored portion of the mask layer 119. The two arrows on the mask layer 119 may be used to position the test strip 110 in a container, and may be used to indicate the end of the test strip 110 for receiving the biological sample. In one example, the test strip 110 is received in a receptacle with a sealed end and an open end. The end of the test strip 110 having the sample pad 113 is disposed at the open end of the receptacle, and the arrows on the mask layer 119 are directed to the open end of the receptacle.

In one embodiment, the device 100 further includes a container (not shown) configured to place one or more test strips 110, and to accommodate the biological sample to be tested. The device may be reusable or disposable.

In one example, the biological sample to be tested is saliva or other body fluid of a patient, and the test strip 110 is configured to evaluate the presence, the amount or concentration of human CD44 in the biological sample, or various forms of a human CD44, or a protein that is homologous to human CD44. CD44 molecule is a cell surface glycoprotein involved in cell-cell interaction, cell adhesion and migration. In human, the CD44 antigen is encoded by the CD44 gene on chromosome 11. In certain embodiments, the CD44 is directed to human CD44 protein. In this example, the substrate 111 is formed from G & L plastic-backed cards or double sided adhesive cards. The sample pad 113 is formed from glass fiber Ahlstrom 8964 pad, where the glass fiber Ahlstrom 8964 is pretreated with Pluronic F127 detergent, and the conjugating portion of the sample pad 113 is sprayed with CD44-conjugate solution. The CD44-conjugate solution includes colloidal 526 gold, BMS209-detector CD44 antibody, colloidal gold blocker, and other optional components, such as sucrose, sarkosyl, buffer, etc. The sample pad 115 is formed from Millipore HF120 nitrocellulose membrane. The test line on the sample pad 115 includes BMS209-capture antibody, and the control line on the sample pad 115 includes goat anti-mouse antibody (GAM). The test line or the control line may also include a dye. The sink pad 117 is formed from Ahlstrom 222 absorbent sink pad.

In another example, the test strip 110 is configured to evaluate the amount or concentration of total proteins in the biological sample to be tested. The total protein test strip 110 may include a substrate 111, a sample pad 113, and a total protein pad 115. The substrate 111 may be formed from a G & L plastic-backed card. The sample pad 113 may be formed from a fiber glass Ahlstrom 8964 sample pad. The total protein pad 115 may include a total protein pad obtained from Teco Diagnostics.

In a further example, the above described CD44 test strip and the total protein test strip may be attached back to back to test the amount of the CD44 protein and the amount of the total protein at the same time using the same biological sample.

In certain embodiments, the strip 110 may be formed by disposing the test pad 115 having a first end and a second end on roughly the center of the substrate 111, disposing the sink pad 117 on the substrate 111 such that one end portion of the sink pad 117 covers the second end of the test pad 115, disposing the sample pad 113 on the substrate 111 such that one end portion of the sample pad 113 aligns with the end of the substrate 111, and the other end portion of the sample pad 115 covers the first end of the test pad 115, and disposing the mask layer 119 on the sample pad 113, the test pad 115 and the sink pad 117.

In certain embodiments, multiple test strips 110 are formed at a batch manner, where large sheets or cards of substrate 11, sample pad 113, test pad 115, sink pad 117 and mask layer 119 are disposed or aligned, and then cut into pieces to form multiple strips 110.

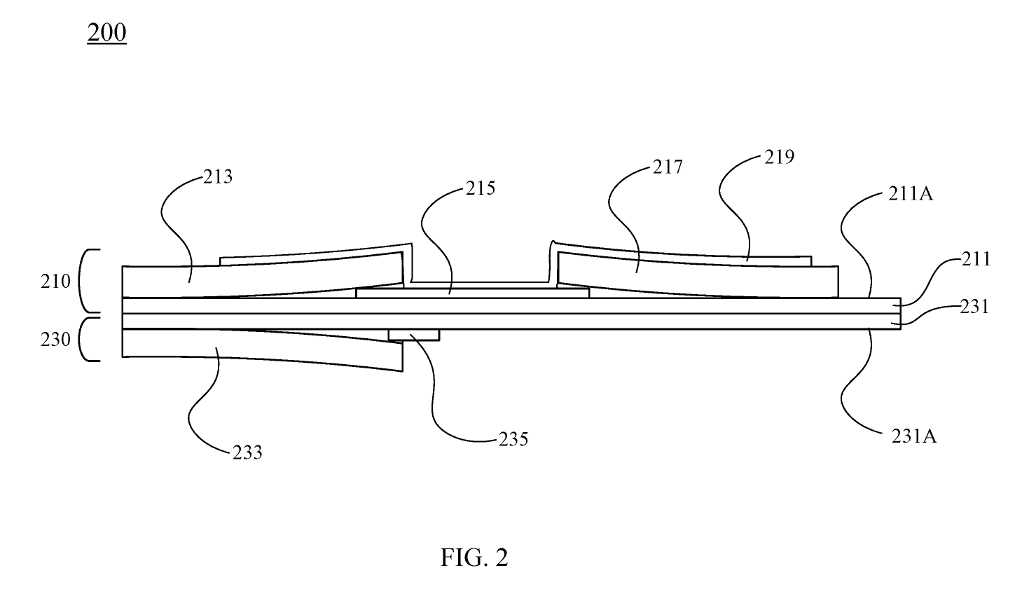

In another aspect, the disclosure relates to a device for testing at least one biological sample. As shown in FIG. 2, a device 200 includes a first test strip 210 and a second test strip 230 attached to each other.

The first test strip 210 of the device 200 may have the same structure as the test strip 110 of the device 100. As shown in FIG. 2, the first test strip 210 includes a first substrate 211, a first sample pad 213, a first test pad 215, a first sink pad 217, and a first mask layer 219. The substrate 211 has a first surface 211A for disposing the first sample pad 213, the first test pad 215, the first sink pad 217 and the first mask layer 219.

The second test strip 230 may or may not have the same structure as the first test strip 210. In certain embodiments, the second test strip 230 includes a second substrate 231, a second sample pad 233 and a second test pad 235, but does not include a sink pad or mask layer. In certain embodiments, the second sample pad 233 is aligned with the first sample pad 213. The length of the second sample pad 233 may be substantially the same as the length of the first sample pad 213. The length of the second test pad 235 may be shorter or the same as the length of the first test pad 215. In certain embodiment, the second substrate 231 has a length of about 20-300 mm, and a width of about 1-20 mm. In one embodiment, the second substrate 231 has a length of about 40-160 mm, and a width of about 3-10 mm. In one embodiment, the second substrate 231 has a length of about 80 mm, and a width of about 4.7 mm. The second substrate 231 has a first surface and an opposite, second surface. The second sample pad 233 is disposed on the first surface 231A of the second substrate 231 and configured to receive the biological sample. In certain embodiment, the second sample pad 233 has a length of about 8-120 mm, and a width of about 1-20 mm. In one embodiment, the second sample pad 233 has a length of about 15-60 mm, and a width of about 3-10 mm. In one embodiment, the second sample pad 233 has a length of about 30 mm, and a width of about 4.7 mm. In one embodiment, the second test pad 235 has a length of about 1-20 mm, and a width of about 1-20 mm. In one embodiment, the second test pad 235 has a length of about 2-10 mm, and a width of about 3-10 mm. In one embodiment, the second test pad 235 has a length of about 5 mm, and a width of about 4.7 mm. The second test pad 235 is disposed on the first surface 231A of the second substrate 231, has a portion in contact with the second sample pad 233, and is configured to test the biological sample received from the second sample pad 233. In one embodiment, the second sample pad 233 is made of a glass fiber Ahlstrom 8964 pad. In one embodiment, the second test pad 235 is a total protein (TP) pad configured to test total protein amount in the sample received from the second sample pad 233. In one embodiment, the TP pad 235 is a Teco Diagnostics TP pad or equivalent. In one embodiment, the color of the TP pad 235 is an indicative of the amount of total protein. In one embodiment, one end of the second sample pad 233 in contact with the second test pad 235 covers one end of the second test pad 235, and the second sample pad 233 overlaps the second test pad 235 by about 0.5-6 mm. In one embodiment, the overlap is about 1-3 mm. In certain embodiments, the second test strip 230 does not have a test line and a control line. The total protein is evaluated by the color change or color intensity of the sample pad.

In certain embodiments, each of the first substrate 211 and the second substrate 231 is made of a plastic-backed card, and the first test strip 210 and the second test strip 230 are assembled back to back. In one embodiment, the back surface of the first substrate 211 and the back surface of the second substrate 231 face to each other, and attached to each other.

In one embodiment, the first test strip 210 is configured to evaluate an amount of CD44 proteins in the biological sample, and the second test strip 230 is configured to evaluate an amount of total proteins in the biological sample. The biological sample to be tested may be saliva or other suitable body fluid, and the evaluation of the amount of CD44 and the total protein in the saliva may be used to detect head and neck squamous cell carcinoma (HNSCC) or monitor states of the HNSCC of a patient.

In certain embodiments, the device 200 of the disclosure may be used for early detection or diagnosis of cancer or cancer risks, where a first test strip 210 is configured to detect presence or quantity of CD44 protein and the second test strip 230 is configure to detect presence or quantity of total protein. In certain embodiments, a line intensity chart is used for comparing with the color of the test line of the first test strip 210 to evaluate the concentration of CD44 protein, and a colored chart is used for comparing with the color of the test pad of the second test strip 230 to evaluate the concentration of the total protein. The result may be used to early detect HNSCC or evaluate risks of HNSCC. In one embodiment, the result may be used to detect the risk of HNSCC occurrence. In one embodiment, the result may be used to evaluate success of a HNSCC treatment. In one embodiment, the result may be used to predict the recurrence of a HNSCC after successful treatment of the HNSCC. In certain embodiments, the test may be used for evaluate cancers other than HNSCC, or diseases other than cancers, where the target molecules to be tested may be CD44 and total protein, or other suitable target molecules.

In a further example, the disclosure is directed to a kit for early detection of cancer risks. The kit includes a first test strip for detection of CD44 protein and a second test strip for detection of total protein. The kit further includes a straw with a sealed end and an open end, for receiving the first test strip and the second test strip, and for receiving biological samples to be tested.

In one example, the test strips are produced in batch, and one or more of the test strips produced in one batch may be tested using a standard solution, for quality control of the produced test strips.

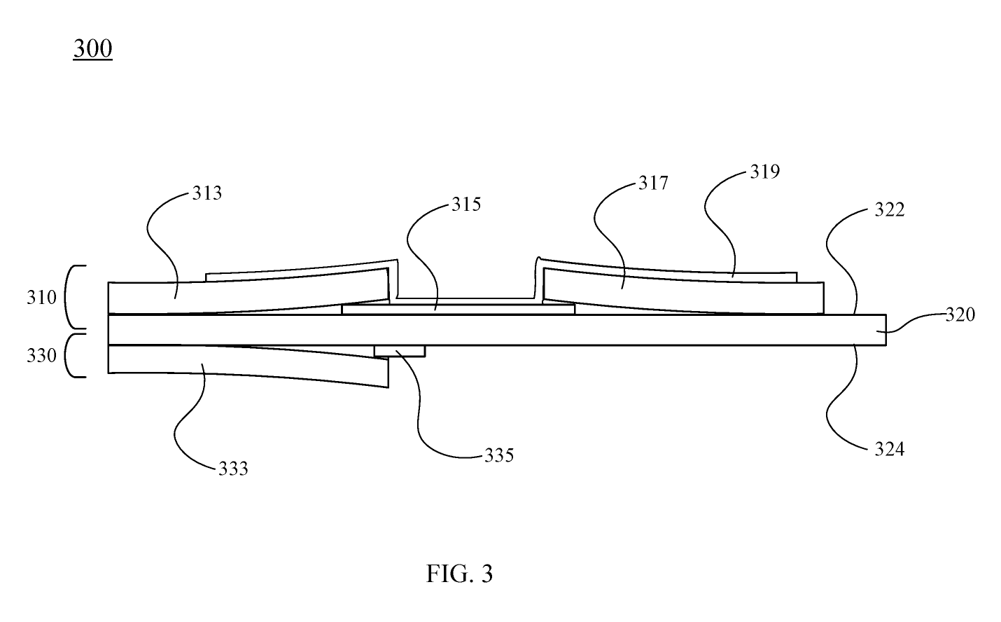

In certain embodiments, the first substrate and the second substrate are one plastic card with double sided adhesive. As shown in FIG. 3, the device 300 includes a first test strip 310 and a second strip 330 share a double sided substrate 320. The double sided substrate 320 has a top surface 322 and a bottom surface 324. The first test strip 310 includes the first sample pad 313, the first test pad 315, the first sink pad 317, and the first mask layer 319 disposed on the top surface 322 of the double sided substrate 320. The second test strip 330 includes the second sample pad 333 and the second test pad 335 disposed on the bottom surface 324 of the double sided substrate 320. Thus, the structure of the device 300 is similar to the device 200, except that the device 300 has a double sided substrate 320 in the place of the two substrates 211 and 231.

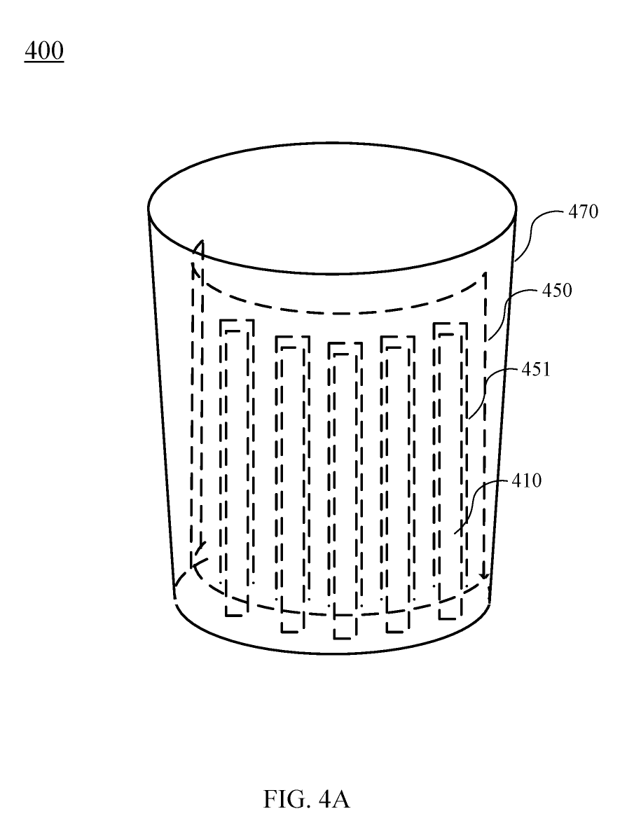

In a further aspect, the disclosure relates to a device for testing at least one biological sample. As shown in FIG. 4A and FIG. 4B, the device 400 includes test strips 410, a support member 450 having multiple notched receptacles 451, and a container 470.

Referring to FIG. 4A, the container 470 has a side wall and a bottom wall. The side wall is in a tubular shape, and defined by an upper diameter corresponding to a cap (fixable or screwable on the container 470, not shown) and a lower diameter corresponding to the circumference of the bottom wall. The upper diameter may be equal to or greater than the lower diameter. The inner wall of the container 470 may include positioning columns for positioning or aligning of the support member 450. The support member 450 may be placed along the inner side of the side wall of the container 470. In certain embodiments, the container 470 may be a transparent cup, such that the support member 450 and the test strips 410 are visible through the transparent wall of the container 470.

Referring to FIG. 4B, the support member 450 is flexible and includes multiple notched receptacles 451 connected side by side, such that the support member 450 has a rectangular plate form. The notched receptacle 451 may have a sealed top end and an open bottom end. Each notched receptacles 451 may hold one test strip 410. In certain embodiment, when the support member 450 is placed in the container 470, the sealed end of each notched receptacle 451 is positioned toward the top of the container 470.

In certain embodiment, the device 400 may include other forms of the receptacles for receiving the test strips. An air chamber is formed in the receptacle, so as to avoid flooding of the sample in the receptacles.

The test strip 410 may have the same structure as the test strip 110 for testing a feature of the biological sample to be tested, or have the same structure as the test strips 210/230 for testing two different features of the biological sample to be tested. As shown in FIG. 4B, the test strips 410-1, 410-2, . . . 410-N are respectively received in the notched receptacles 451-1, 451-2, . . . , 451-N of the support member 450. In certain embodiments, the test strips 410 are slidably received in the notched receptacles 451. The test strips 410 may be labeled with a marker to show the type of the test strip 410 or to indicate the testing the test strip can perform. The marker may be a short tape attached to the top and back surface of the substrate of the test strip 410. The marker is located at the end of the strip 410 having the sink pad, and is located at the side of the substrate opposite to the side where the sink pad is disposed. The length of the test strip 410 is slightly greater than the depth of the notched receptacle 451, such that a small portion of the end of the testing strip 410 is exposed from the open end of the corresponding notched receptacle 451. In one embodiment, one end of the test strip 410 with sample pad is exposed from the notched receptacle 451. In one embodiment, the sample pad is exposed about 0.5-8 mm from the open end of the corresponding notched receptacle 451. In one embodiment, the sample pad is exposed about 1-4 mm from the open end of the corresponding notched receptacle 451. In one embodiment, the sample pad is exposed about 2 mm from the open end of the corresponding notched receptacle 451.

In certain embodiments, the device 400 may further include at least one color chart (not shown) used as a reference to the color of the test line of the strip 410. In one example, the color chart may be a label or instruction fixed on the wall surface of the container 470 for explanation of the test result. In one embodiment, a positive or a negative result corresponds to two colored lines revealed on a corresponding test strip, where a C line indicates the control line and a T line indicates the test line. In one embodiment, a negative or a positive result corresponds to one colored C line revealed on a corresponding test strip, where T line cannot be seen. Whether two colored lines or one colored line correspond to a positive result or a negative result depends on the chemistry used in the testing. In one embodiment, showing of two lines (control line and test line) is positive, and showing of one line (control line) is negative. In one embodiment, showing two lines (control line and test line) is negative, such as a drug test employing a competitive binding. In one embodiment, neither the C line nor the T line can be seen, which may reveal a test failure.

In certain embodiments, each of the multiple test strips 410 may be used to test at least one feature different from features tested by any other test strips 410, and the multiple strips 410 may be used to test one biological sample and provide result of multiple features for the biological sample. Under this situation, the container 470 may be used to receive the biological samples and all the tests by the test strips 410 may be performed at once.

In certain embodiments, the test strips 410 may include several types of test strips, and each type includes one or more test strips. The one or more test strips with the same type have the same structure and are configured to test at least one same feature of a biological sample. Under this situation, a few test strips belong to different types may be chosen from the multiple strips 410 to test the biological sample, and the other unused test strips 410 may be used later for other tests.

In certain embodiments, when in operation, the support member 450 with the multiple test strips 410 is pulled out from the container 470. A biological sample, such as 5 milliliters of saliva of a patient, may be added to the container 470. Then the support member 450 with multiple test strips 410 are inserted back into the container 470 such that part of the sample pads of the multiple test strips 410 are immersed in the biological sample. The support member 450 with the multiple test strips 410 are kept in the biological sample for a period of time, and the testing results can be evaluated for each test strip 410.

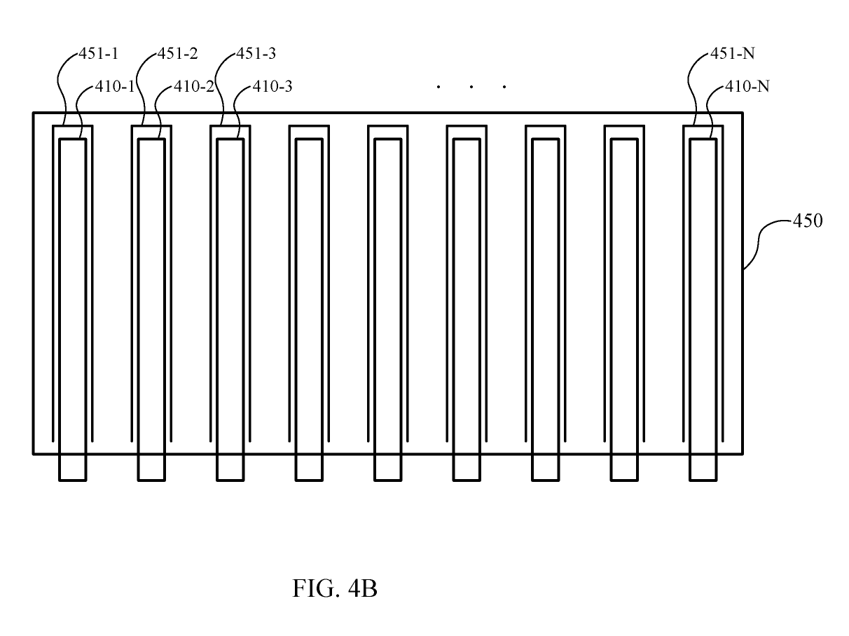

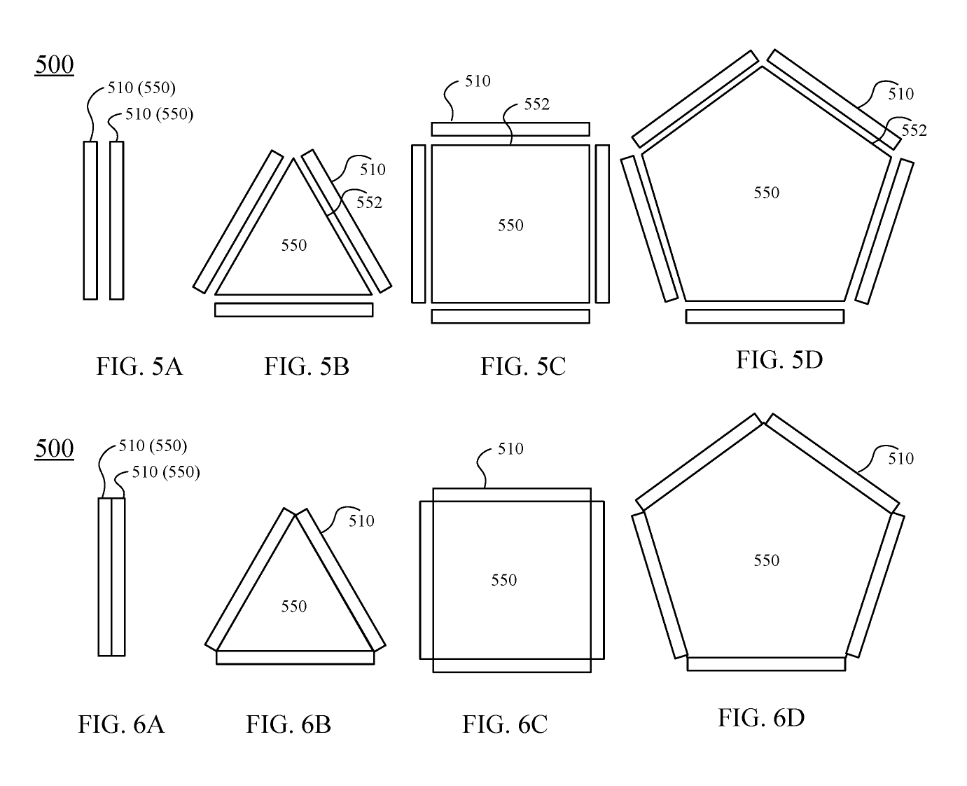

In yet another aspect, the disclosure relates to a device for testing at least one biological sample. As shown in FIGS. 5A-6D, each device 500 includes multiple test strips 510 and a support member 550. FIGS. 5A-5C are sectional views of the device 500, where the test strips 510 are not attached to the support member 550. FIGS. 6A-6D are sectional views of the device 500, where the test strips 510 are attached to the support member 550.

Each of the test strips 510 may have the same structure as the test strip 110 for testing a feature of the biological sample to be tested. In certain embodiments, the test strips 510 in one device 500 may be configured to test different features of the biological sample, such that the device 500 may be used upon one biological sample, and the different features of the biological sample can be obtained by one test. In other embodiments, two or more test strips 510 may be configured to test the same feature of a biological sample, and each of the test strips 510 attached to one support member 550 may be detached and used separately.

As shown in FIG. 5A, the substrate of the test strip 510 may be function as the support member 550, and the two test strips 510 may be attached back to back to form a dual test strip device as shown in FIG. 5A.

As shown in FIG. 5B and FIG. 6B, the support member 550 is a triangular prism with three side surfaces 552. The cross-sectional view of the support member 550 is an equilateral triangle. The three test strips 510 are each attached to one of the side surfaces 552 to form the triangular prism shaped device 500. The tree test strips 510 may be configured to test different features of a biological sample, or two or more strips 510 may be configured to test the same features of the biological sample. As shown in FIG. 5C and FIG. 6C, the support member 550 is a square prism with four side surfaces 552. The cross-sectional view of the support member 550 is a square. The four test strips 510 are each attached to one of the side surfaces 552 to form the square prism shaped device 500. The four test strips 510 may be configured to test different features of a biological sample, or two or more strips 510 may be configured to test the same features of the biological sample. As shown in FIG. 5D and FIG. 6D, the support member 550 is a pentagonal prism with five side surfaces 552. The cross-sectional view of the support member 550 is a regular pentagon. The five test strips 510 are each attached to one of the side surfaces 552 to form the pentagonal prism shaped device 500. The five test strips 510 may be configured to test different features of a biological sample, or two or more strips 510 may be configured to test the same features of the biological sample.

In certain embodiments, the support member 550 may be a prism having more than five side surfaces 552, such that more than five test strips 510 may be attached to the side surfaces 552 of the prism. In certain embodiments, the support member 550 may be an irregular prism, where the side surfaces 552 of the support member 550 may not be identical. For example, the width of one side surface 552 may be smaller than the width of the other side surface 552, and the width of each side surfaces 552 is sufficient to meet the requirement of certain type of test strips 510. In certain embodiment, the support member 550 of the device 500 is in a shape of a thin plate, a prism or any other geometric form that is suitable for the packaging or assembling of the test strips 510.

In certain embodiments, before assembling the test strips 510 and the support member 550, the test strips 510 are not separated. Instead, the test strips 510 are integrally formed as one piece, for example, the substrate of all the test strips is one whole piece. Then, the test strips 510 formed as one piece are attached by surrounding the support member 550 and adhered to the side surfaces 552 of the support member 550, such that each test strip 510 corresponds to a corresponding side surface 552.

In certain embodiments, the support member 550 may be a solid structure or a hollow structure surrounded by the side surfaces 552. In one embodiment, the hollow structure may reduce the weight of the device while maintain the strength of the device. When the support member 550 has a hollow structure, each side surface of the support member 550 may include an outer surface facing outward and an inner surface facing inward. One test strip 510 may be disposed on the outer surface of a side surface, and the other test strip 510 may be disposed on the inner surface of that side surface.

In certain embodiments, the support member 550 may be made of plastic material, paper, or polymer. In one embodiment, the support member is made of light weight, inert materials that do not interfere the reaction between the test strips 510 and the sample.

In certain embodiment, the test strips 510 are attachable and releasable from the side surfaces 552 of the support member 550 of the device 500. In certain embodiments, the support member 550 is a reusable structure and the test strips 510 are attachable and releasable from the support member 550. A user can choose specific test strips 510 to be attached onto the support member 550 before performing a targeted test. The combination of the strips 510 can be determined by the user or suggested by a professional person in the field. In operation, all the side surfaces 552 of the support member 550 may not be attached with a test strip 510.

In certain embodiments, the substrate of each test strips 510 may be function as the support member 550, and the substrates of the test strips 510 may be connected or integrally formed as the support member 550. Under this situation, the support member 550 may have a hollow structure. In one embodiment, each of the test strips 510 may have a structure of the test strip 110 or the test strips 210/230 or the test strips 310/330 as shown in FIGS. 1-3.

In one embodiment, the device further includes a container configured to accommodate at least one of the support member 550 with the test strips 510 and the biological sample. In one embodiment, the device is reusable or disposable.

In certain embodiments, the device 500 of the disclosure can be used for early detection or diagnosis of cancer or cancer risks, where one test strip 510 is configured to detect presence or quantity of CD44 protein and the other test strip 510 is configure to detect presence or quantity of total protein. The result may be used to early detect or evaluate risks of HNSCC. In one embodiment, the result may be used to detect the risk of cancer occurrence. In one embodiment, the result may be used to evaluate success of a cancer treatment. In one embodiment, the result may be used to predict the recurrence of a cancer after successful treatment of the cancer.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the disclosure pertains without departing from its spirit and scope. Accordingly, the scope of the disclosure is defined by the appended claims as well as the disclosure including drawings.

What is claimed is:

1. A device for testing a biological sample, comprising: a test strip, wherein the test strip comprises:
a substrate having a first surface and an opposite, second surface;
a first sample pad and a second sample pad disposed on the first surface and the second surface of the substrate respectively, and configured to receive the biological sample;
a first test pad disposed on the first surface of the substrate, in contact with the first sample pad, and configured to test the biological sample received from the first sample pad; and
a second test pad disposed on the second surface of the substrate, in contact with the second sample pad, and configured to test the biological sample received from the second sample pad.

2. The device of claim 1, wherein the first test pad is configured to determine presence or a concentration of a target molecule of interest in the biological sample, and the second test pad is configured to determine presence or a concentration of total protein in the biological sample.

3. The device of claim 2, wherein the target molecule of interest is a biomarker for indicating a biological state or condition.

4. The device of claim 3, wherein the biomarker comprises CD44 or a protein homologous to CD44.

5. The device of claim 2, wherein the presence of the target molecule of interest and the presence of the total protein in the biological sample are determined by visually viewing the first test pad and the second test pad respectively.

6. The device of claim 2, wherein the concentration of the target molecule of interest and the concentration of the total protein in the biological sample are determined using an electronic reader or a reflectance reader.

7. The device of claim 2, wherein
the first test pad comprises a test line and a control line, the device further comprises a target intensity chart and a total protein intensity chart;
the target intensity chart including a relationship between a color intensity of the test line and the concentration of the target molecule of interest, and the concentration of the target molecule of interest in the biological sample is determined by comparing the color intensity of the test line with the target intensity chart; and
the total protein intensity chart including a relationship between a color intensity of the second test pad and the concentration of the total protein, and the concentration of the total protein in the biological sample is determined by comparing the color intensity of the second test pad with the total protein colored chart.

8. The device of claim 1, wherein the test strip further comprises a first sink pad disposed on the first surface of the substrate such that the first test pad is placed between the first sample pad and the first sink pad.

9. The device of claim 1, further comprising a container configured to accommodate the biological sample, wherein the test strip is placed in the container for receiving the biological sample.

10. The device of claim 1, wherein
the substrate is flexible and formed of plastic, metal, or glass; and
at least one of the first test pad and the second test pad is formed of a nitrocellulose membrane, a nylon membrane, or an activated cellulose membrane.

11. The device of claim 1, wherein the substrate comprises a first substrate sheet and a second substrate sheet attached back to back, such that the first substrate sheet exposes the first surface and the second substrate exposes the second surface.

12. A device for testing at least one biological sample, comprising:
a support member having a plurality of side surfaces; and
a plurality of test strips, each attached to one of the plurality of side surfaces, wherein at least one of the test strips comprises:
a sample pad, configured to receive the biological sample; and
a test pad in contact with the sample pad, and configured to test the at least one biological sample received from the sample pad.

13. The device of claim 12, wherein the support member is in a shape of prism comprising triangular prism, square prism, pentagonal prism, and a prism having more than five side surfaces.

14. The device of claim 13, wherein the support member has a hollow structure, such that each of the side surfaces comprises an outer surface facing outward and an opposite, inner surface facing inward.

15. The device of claim 14, wherein at least one of the side surfaces has a first test strip of the plurality of test strips disposed on the outer surface of the at least one of the side surfaces, and a second test strip of the plurality of test strips disposed on the inner surface of the at least one of the side surfaces.

16. The device of claim 15, wherein the first test strip is configured to determine presence or a concentration of CD44 protein in the at least one biological sample, and the second test strip is configured to determine presence or a concentration of total protein in the at least one biological sample.

17. The device of claim 12, wherein
a first test strip of the plurality of test strips is disposed on at least one surface of the side surfaces, and configured to determine presence or a concentration of CD44 protein in the at least one biological sample; and
a second test strip of the plurality of test strips is disposed on at least the other one of the side surfaces, and configured to determine presence or a concentration of total protein in the at least one biological sample.

18. The device of claim 12, wherein the support member comprises a plurality of notched receptacles, each notched receptacle having a sealed end and an open end, and each of the test strips is slidably received in one of the notched receptacles through the open end thereof.

19. The device of claim 12, wherein
at least one of the test strips further comprises a substrate attached to one of the side surfaces; and
at least one of the test strips further comprises at least one sink pad in contact with the test pad of the at least one of the test strips.

20. The device of claim 19,
wherein the substrate is flexible and formed of plastic, metal, or glass; and
wherein the test pad is formed of a nitrocellulose membrane, a nylon membrane, or an activated cellulose membrane.

21. The device of claim 12, further comprising a container configured to accommodate the at least one biological sample, wherein the support member and the test strips are placed in the container for receiving the biological sample.

22. The device of claim 12, wherein at least one of the test strips is received in an air chamber of the support member, such that flooding of the biological sample is avoided in the air chamber.
